(19) United States
(12) Patent Application Publication
(10) Pub. No.: US 2023/0147124 A1
(43) Pub. Date: May 11, 2023

(54) PLACEMENT DEVICE FOR AN INFLATABLE INTRA-GASTRIC BALLOON SYSTEM FOR TREATING OBESITY IN AN INDIVIDUAL COMPRISING SUCH A PLACEMENT DEVICE

(71) Applicant: MEDICAL INNOVATION DEVELOPPEMENT, DARDILLY (FR)

(72) Inventors: Ludovic CAZENAVE, LYON (FR); Yannis CLAIR, LA VERPILLIERE (FR)

(21) Appl. No.: 17/916,317

(22) PCT Filed: Mar. 31, 2021
(86) PCT No.: PCT/FR2021/050569

§ 371 Date: Sep. 30, 2022

(30) Foreign Application Priority Data
Apr. 2, 2020 (FR) 20 03327

(51) Int. Cl. A61F 5/00

(52) U.S. Cl. CPC A61F 5/0043; A61F 5/003; A61F 5/0089

(57) ABSTRACT

A device for implanting an inflatable intra-gastric balloon for implanting the intra-gastric balloon into a stomach, comprising: —an elastically deformable sheath configured to receive the intra-gastric balloon in the deflated state, the sheath comprising a side wall, —a tube configured to be supplied with a fluid and to cause the fluid to inflate the intra-gastric balloon, the tube comprising a mouthpiece, the intra-gastric balloon being attached to the mouthpiece in the deflated state and being detached from the mouthpiece in the inflated state, the mouthpiece having an inflation position wherein the mouthpiece extends in the cavity of the sheath, and the side wall of the sheath having a side opening configured to release the intra-gastric balloon from the cavity when the intra-gastric balloon moves from the deflated state to the inflated state.

### TECHNICAL FIELD

The present invention relates to a placement device for an inflatable gastric balloon intended to be inserted into the stomach of an individual and to a system for treating obesity comprising such a placement device.

### PRIOR ART

For the past few years, there has been an increase in obesity in the world population. One of the known treatments for treating individuals suffering from obesity is to fit an inflatable gastric balloon in the stomach.

The gastric balloon may notably be introduced endoscopically. The deflated gastric balloon is positioned at the end of a duct, for example a catheter, and the duct is then inserted into the mouth and then the oesophagus of the individual suffering from obesity until the gastric balloon reaches the stomach of the individual. The gastric balloon is then inflated with physiological serum, air or a mixture of the two, conveyed via the duct.

Once inflated, the gastric balloon is detached from the duct and remains implanted in the stomach, notably at the fundus.

This treatment is easy to perform and minimally invasive. However, it would be appropriate to find a system that allows better monitoring of the detachment of the gastric balloon from the duct, and makes it possible to ensure that the gastric balloon is correctly positioned in the stomach, for example in the fundus.

### SUMMARY

The present invention seeks to meet the abovementioned need.

To this end, according to a first aspect, the invention provides a placement device for an inflatable gastric balloon for the placement of the gastric balloon in a stomach, the gastric balloon having a deflated state and an inflated state, the placement device comprising:
an elastically deformable casing delimiting a cavity configured to receive the gastric balloon in the deflated state, the casing extending along a longitudinal axis and comprising a lateral wall extending around the longitudinal axis between a connection end and an insertion end,
a duct configured to be supplied with a fluid and to convey the fluid to inflate the gastric balloon, the duct being attached to the connection end of the casing, and comprising an end piece configured to removably attach the gastric balloon, characterized in that the end piece has an inflation position in which said end piece extends into the cavity of the casing,
   and in that the lateral wall of the casing has a lateral opening configured to allow the gastric balloon to exit the cavity when said gastric balloon passes from the deflated state to the inflated state.

Thus, the collaboration between the end piece of the duct and the gastric balloon ensures simplified detachment of the gastric balloon from the duct.

In addition, the lateral opening ensures correct positioning of the gastric balloon in the stomach when the gastric balloon is detached from the duct. This is because the lateral opening provides control over the direction in which the gastric balloon is released into the stomach, and thus allows it to be guided towards the fundus.

The placement of the gastric balloon in the stomach is thus facilitated.

According to a variant of the invention, the duct comprises a sheath attached to the connection end of the casing and a catheter extending inside the sheath, the catheter being mounted with the ability to slide with respect to the sheath and comprising a proximal end configured to be supplied with fluid and a distal end, the distal end comprising the end piece and projecting from the sheath in the inflation position.

The catheter can slide in the sheath to cause the end piece to exit the cavity, thus allowing the gastric balloon to be positioned on the end piece when the latter is outside of the cavity. The fitting of the gastric balloon to the end piece is therefore facilitated. In addition, the catheter can also slide in the sheath in order to allow the gastric balloon to be detached from the end piece.

According to a variant of the invention, the lateral wall of the casing has a bearing surface, opposite the lateral opening and configured so that the gastric balloon bears against said bearing surface when said gastric balloon passes from the deflated state to the inflated state.

The bearing surface thus guides the gastric balloon towards the lateral opening.

According to a variant, the lateral opening of the casing has a bearing edge configured so that the gastric balloon bears against said bearing edge for detaching the gastric balloon from the end piece.

The gastric balloon can thus be detached from the end piece without the gastric balloon bearing against the wall of the stomach.

According to a variant, in the inflation position, the end piece extends along the longitudinal axis and is positioned between the connection end of the casing and the lateral opening of the casing along the longitudinal axis.

This position of the end piece makes it possible to optimize the positioning of the gastric balloon in the cavity when the balloon passes from the deflated state to the inflated state, so as to allow the gastric balloon to exit the casing automatically via the lateral opening.

According to a variant, the placement device is specially designed for a gastric balloon comprising a valve configured to collaborate with the end piece of the duct and having an open state allowing fluid to pass and a closed state blocking the fluid, and the end piece is configured to be push-fitted removably into the valve of the gastric balloon.

The push-fitting makes it easier for the gastric balloon to be positioned on the end piece in the deflated state while at the same time ensuring the detachment of the gastric balloon in the inflated state.

Where appropriate, the end piece may have an end piece axis and comprise at least one shoulder extending transversely with respect to the end piece axis.

The shoulder contributes to holding the gastric balloon on the end piece in the deflated state and when the gastric balloon is passing from the deflated state to the inflated state, while at the same time allowing the detachment of the gastric balloon in the inflated state.

According to a variant, the lateral opening has a shape that is oblong along the longitudinal axis.

According to a variant, the placement device is specially designed for a spherical gastric balloon having a diameter in the inflated state, and the cavity of the casing and the lateral opening each have a length considered along the longitudinal axis and a width considered perpendicular to the longitudinal axis such that:
a ratio of the diameter of the gastric balloon to the length of the cavity of the casing is comprised between 0.44 and 0.83,
a ratio of the diameter of the gastric balloon to the width of the cavity of the casing is comprised between 3.2 and 6.7,
a ratio of the length of the lateral opening of the casing to the length of the cavity of the casing is comprised between 0.17 and 0.67,
a ratio of the width of the lateral opening of the casing to the width of the cavity of the casing is comprised between 0.24 and 0.67.

According to a variant, the placement device is specially designed for a spherical gastric balloon having a diameter comprised between 8 cm and 10 cm in the inflated state, and:
the length of the cavity of the casing is comprised between 12 cm and 18 cm, and the width of the cavity of the casing is comprised between 1.5 cm and 2.5 cm,
the length of the lateral opening of the casing is comprised between 3 cm and 8 cm and the width of the lateral opening of the casing is comprised between 0.6 cm and 1.0 cm.

According to a variant, the casing is a cylinder along the longitudinal axis, of circular cross section.

This shape of the casing limits the risk of creating lesions in the natural tracts of the human body into which the casing is inserted.

According to a variant, the insertion end of the casing is convex towards the outside of the cavity, preferably hemispherical, so that the placement device can be introduced into a natural tract of the human body.

This convex, or even hemispherical, shape of the casing makes the casing easier to insert into the natural tracts of the human body while limiting the risk of lesions.

According to a second aspect of the invention, the invention proposes a system for treating obesity in an individual, comprising:
a placement device according to the first aspect of the invention, and
an inflatable gastric balloon having an inflated state and a deflated state.

According to a variant, the gastric balloon is made of silicone. Silicone allows the gastric balloon to deform without tearing.

According to a variant, the gastric balloon comprises a valve configured to collaborate with the end piece of the duct and having an open state allowing fluid to pass and a closed state blocking the fluid.

In the open state, the valve allows fluid to pass from the duct into the gastric balloon, thus allowing the gastric balloon to pass from the deflated state to the inflated state. In the closed state, the valve prevents the fluid situated in the gastric balloon from leaving the gastric balloon. The valve may for example be a one-way valve and have a direction that allows flow, in which the direction the fluid can circulate from the duct into the gastric balloon, and a direction that blocks flow, in which direction it prevents the fluid from circulating, preventing the fluid situated in the gastric balloon from leaving the gastric balloon.

The invention can be employed in a method for treating obesity in an individual.

In particular, the treatment system according to the second aspect of the invention can be employed in a method for treating obesity in an individual, wherein
the gastric balloon in the deflated state is attached to the end piece of the duct of the placement device,
the casing of the placement device is inserted into the stomach of the individual,
the duct is supplied with a fluid and the duct conveys the fluid to inflate the gastric balloon,
the gastric balloon exits the casing inside the stomach via the lateral opening in the lateral wall of the casing, and is detached from the end piece in the inflated state,
the duct of the placement device is withdrawn from the stomach of the individual.

According to a variant, in order to attach the gastric balloon in the deflated state to the end piece of the duct, the catheter is slid in the sheath in order to cause the end piece to exit the cavity of the casing.

According to a variant, the gastric balloon bears on the bearing surface of the lateral wall of the casing when the gastric balloon passes from the deflated state to the inflated state.

According to a variant, the gastric balloon automatically exits the casing via the lateral opening when the gastric balloon passes from the deflated state to the inflated state.

According to a variant, the casing of the placement device is inserted via the insertion end into a natural tract of the body of the individual.

### BRIEF DESCRIPTION OF THE DRAWINGS

Further features, details and advantages will become apparent from reading the following detailed description and from studying the attached drawings, in which:

FIG. 1 shows a system for treating obesity in an individual according to the invention comprising a placement device comprising a casing and an inflatable gastric balloon, the gastric balloon being positioned in the casing in a deflated state;

FIG. 2 shows the placement device of the system for treating obesity of FIG. 1;

FIG. 3 shows the gastric balloon of the system for treating obesity of FIG. 1, in the deflated state;

FIG. 4 shows the gastric balloon of the system for treating obesity of FIG. 1 in the inflated state;

FIG. 5 shows the system for treating obesity of FIG. 1 comprising a duct comprising a sheath and a catheter sliding in the sheath, and in which the catheter has been slid in the sheath so that the end piece exits the cavity of the casing;

FIG. 6 shows an enlarged view of the region referenced VI in the system for treating obesity of FIG. 1;

FIG. 7 shows the system for treating obesity of FIG. 1 in which the placement device comprises an end piece, the end piece being in an inflation position, and in which the gastric balloon is attached to the end piece, is between the deflated state and the inflated state and partially emerges from the casing via a lateral opening therein;

FIG. 8 shows the system for treating obesity of FIG. 1 in which the gastric balloon is detached from the end piece and is in the inflated state.

### DESCRIPTION OF THE EMBODIMENTS

In the figures, the same references refer to elements that are identical or analogous.

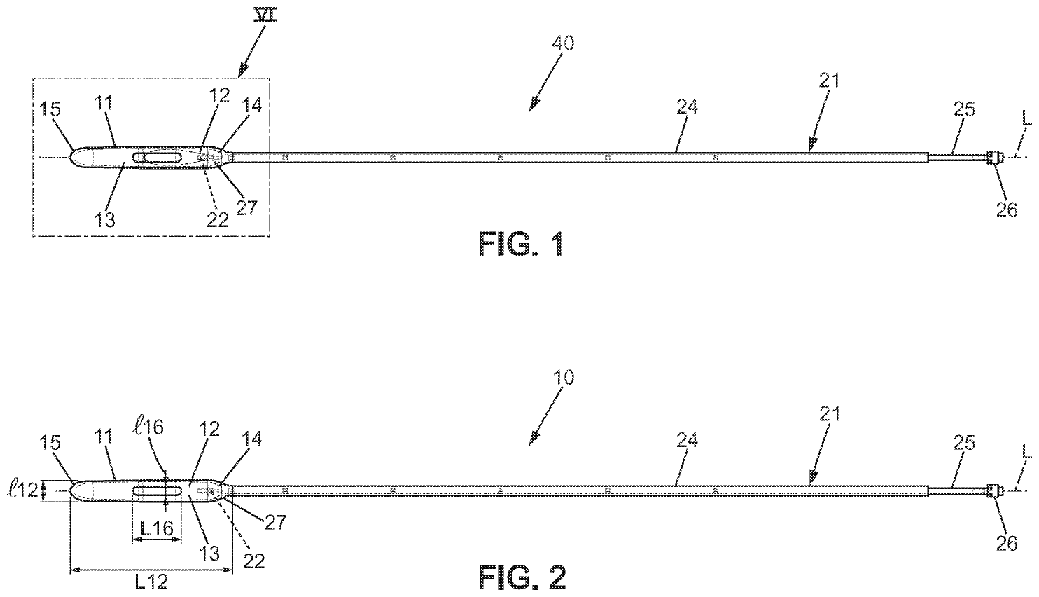

FIG. 1 depicts a system 40 for treating obesity in an individual, comprising a placement device 10 and an inflatable gastric balloon 30.

The placement device 10 allows the placement of the inflatable gastric balloon 30 in the stomach of the individual. In particular, placement may be done endoscopically, via a natural tract of the body of the individual, such as, for example, via their mouth and their oesophagus.

The gastric balloon 30 is made of silicone. Alternatively, it could be made of any other elastically deformable and biocompatible material.

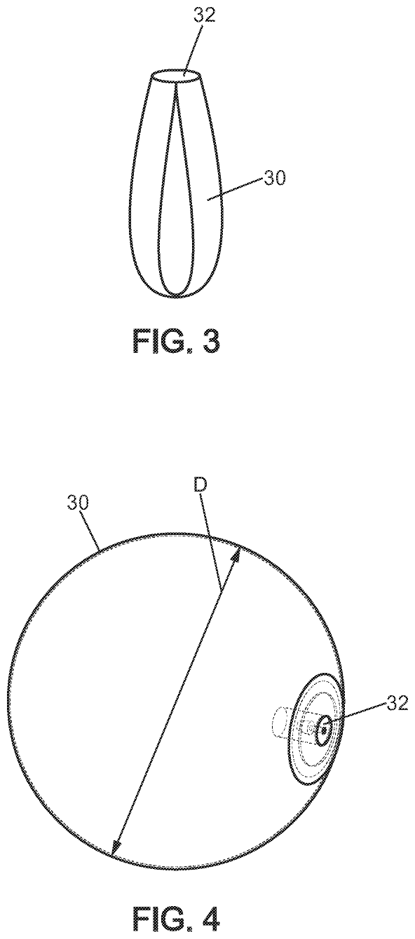

The gastric balloon 30 has a deflated state depicted in FIG. 3 and an inflated state depicted in FIG. 4. The gastric balloon 30 is implanted into the stomach of the individual in the inflated state. The gastric balloon 30 passes from the deflated state to the inflated state by being filled with a fluid such as physiological serum, air or a mixture of physiological serum and air.

The gastric balloon 30 comprises a valve 32 having an open state allowing the fluid to pass and a closed state blocking the fluid. In the open state, the valve 32 allows the fluid to enter the gastric balloon 30, which allows the gastric balloon 30 to be inflated. In the closed state, the valve 32 prevents the fluid situated inside the gastric balloon 30 from leaving the gastric balloon 30. The valve 32 therefore allows the gastric balloon 30 to be kept in the inflated state inside the stomach of the individual.

In the deflated state, the gastric balloon 30 is folded on itself. In the inflated state, the gastric balloon 30 is spherical and has a diameter D. Alternatively, it could adopt an ovoid or ellipsoid shape.

The placement device 10 is depicted in FIG. 2. It comprises a casing 11 and a duct 21. The casing 11 is able to accept the gastric balloon 30, and the duct 21 allows the gastric balloon 30 to be attached and positioned in the casing 11. The duct 21 can be supplied with a fluid and allows the fluid to be conveyed as far as the gastric balloon 30 to inflate the gastric balloon 30.

The casing 11 is elastically deformable. The casing 11 is cylindrical about a longitudinal axis L. It comprises a lateral wall 13 extending about the longitudinal axis between a connection end 14 and an insertion end 15. The cross section of the lateral wall 13 on a plane perpendicular to the longitudinal axis L is a circular cross section. Alternatively, the cross section could be an elliptical cross section or an oval cross section. The casing 11 thus has no sharp edges. When the casing 11 is introduced via a natural tract of the human body, it is thus less liable to cause lesions in this natural tract of the human body.

The insertion end 15 of the casing 11 is hemispherical. Alternatively, it could adopt some other convex shape, for example a portion of a hemisphere. This shape makes the placement device 10 easier to insert endoscopically while limiting the risk of causing lesions in the natural tract of the human body into which the placement device is introduced.

The lateral wall 13 has a lateral opening 16. The lateral opening 16 has a shape that is oblong along the longitudinal axis L. It has two sides that are parallel to one another and to the longitudinal axis L and the same length. Each end of one of the two sides is joined to one of the ends of the other side by an arc of a circle.

The lateral wall 13 has a bearing surface 17, opposite the lateral opening 16 and configured so that the gastric balloon 30 bears upon it when the gastric balloon 30 passes from the deflated state to the inflated state. The bearing surface 17 guides the gastric balloon 30 towards the lateral opening 16.

The casing 11 delimits a cavity 12 able to accept the gastric balloon 30 in the deflated state. As the gastric balloon 30 passes from the deflated state to the inflated state, namely when it is in the process of becoming inflated, the lateral opening 16 of the lateral wall 13 of the casing 11 allows the gastric balloon to exit the cavity.

The cavity 12 of the casing 11 has a length L12 considered along the longitudinal axis L and a width I12 considered perpendicular to the longitudinal axis. The length L12 of the cavity 12 of the casing 11 is considered from the connection end 14 as far as the insertion end 15.

The lateral opening 16 has a length L16 considered along the longitudinal axis L and a width I16 considered perpendicular to the longitudinal axis L. The length L16 of the lateral opening is considered from a vertex S1 of one of the arcs of a circle joining the ends of the two parallel sides of the lateral opening 16 to a vertex S2 of the other arc of a circle joining the ends of the two parallel sides of the lateral opening 16.

The vertex S2 forms a bearing edge against which the gastric balloon 30 can bear so as to allow the gastric balloon 30 to become detached from the duct 21, thus preventing the gastric balloon 30 from pressing against a wall of the stomach of the individual in order to detach itself from the duct 21.

The duct 21 is attached to the connection end 14 of the casing 11. In this example, the duct 21 comprises a sheath 24 attached to the connection end 14 of the casing 11 and a catheter 25 extending inside the sheath 24. The catheter 25 is mounted with the ability to slide with respect to the sheath 24. It comprises a proximal end 26 and a distal end 27 at the opposite end to the proximal end 26. The distal end 27 projects from the sheath 24. In this example, the proximal end 26 of the catheter 25 is supplied with fluid and the catheter 25 conveys the fluid as far as the distal end 27. In another example which has not been depicted, the duct 21 could consist solely of a catheter.

The distal end 27 comprises an end piece 22. The end piece 22 allows the gastric balloon 30 to be attached removably. In particular, in the deflated state, the gastric balloon 30 can be attached to the end piece 22 and in the inflated state, the gastric balloon 30 can be detached from the end piece 22.

The end piece 22 is depicted in an inflation position. The end piece 22 is in this inflation position notably when the gastric balloon 30 passes from the deflated state to the inflated state. In this inflation position, the end piece 22 extends into the cavity 12 of the casing 11. In particular, the end piece 22 extends along the longitudinal axis L and is positioned between the connection end 14 of the casing 11 and the lateral opening 16 of the casing 11 along the longitudinal axis L. This positioning allows the gastric balloon 30 to exit the casing automatically via the lateral opening 16 when the gastric balloon 30 passes from the deflated state to the inflated state.

In order to convey the fluid as far as the gastric balloon 30, the end piece 22 collaborates with the valve 32 of the gastric balloon 30. The end piece 22 is push-fitted removably into the valve 32 of the gastric balloon 30.

The end piece 22 extends along an end piece axis and comprises a shoulder 23 extending transversely with respect to the end piece axis. This shoulder 23 helps with push-fitting the gastric balloon 30 onto the end piece 22 removably. In particular, the gastric balloon 30 can be push-fitted onto the end piece 22 in the deflated state, it can be kept push-fitted on the end piece 22 of the duct 21 during the inflation process, and it can be detached from the end piece 22 of the duct 21 in the inflated state.

By way of illustration, in one nonlimiting particular example, the treatment system is employed with the following dimensions.

A ratio of the diameter D of the gastric balloon 30 to the length L12 of the cavity 12 of the casing 11 may be comprised between 0.44 and 0.83. A ratio of the diameter D of the gastric balloon 30 to the width I12 of the cavity 12 of the casing 11 may be comprised between 3.2 and 6.7. A ratio of the length L16 of the lateral opening 16 of the casing 11 to the length L12 of the cavity 12 of the casing 11 may be comprised between 0.17 and 0.67. A ratio of the width I16 of the lateral opening 16 of the casing 11 to the width I12 of the cavity of the casing could be comprised between 0.24 and 0.67.

The diameter D of the gastric balloon 30 may be comprised between 8 cm and 10 cm. For such a diameter D of the gastric balloon, the length L12 of the cavity 12 of the casing 11 may be comprised between 12 cm and 18 cm, and the width I12 of the cavity 12 of the casing 11 may be comprised between 1.5 cm and 2.5 cm.

Further, the length L16 of the lateral opening 16 may be comprised between 3 cm and 8 cm, and the width I16 of the lateral opening 16 may be comprised between 0.6 cm and 1.0 cm.

In one exemplary embodiment, the ratio of the diameter D of the gastric balloon 30 to the length L12 of the cavity 12 of the casing 11 is equal to 0.6. The ratio of the diameter D of the gastric balloon 30 to the width I12 of the cavity 12 of the casing 11 is equal to 4.5. The ratio of the length L16 of the lateral opening 16 of the casing 11 to the length L12 of the cavity 12 of the casing 11 is equal to 0.3. The ratio of the width I16 of the lateral opening of the casing 11 to the width I12 of the cavity of the casing is equal to 0.4.

In one exemplary embodiment, the diameter D of the gastric balloon 30 is equal to 9 cm. The length L12 of the cavity 12 of the casing 11 is equal to 15 cm and the width I12 of the cavity 12 of the casing 11 is equal to 2 cm. The length L16 of the lateral opening 16 is equal to 4.5 cm and the width I16 of the lateral opening 16 is equal to 0.8 cm.

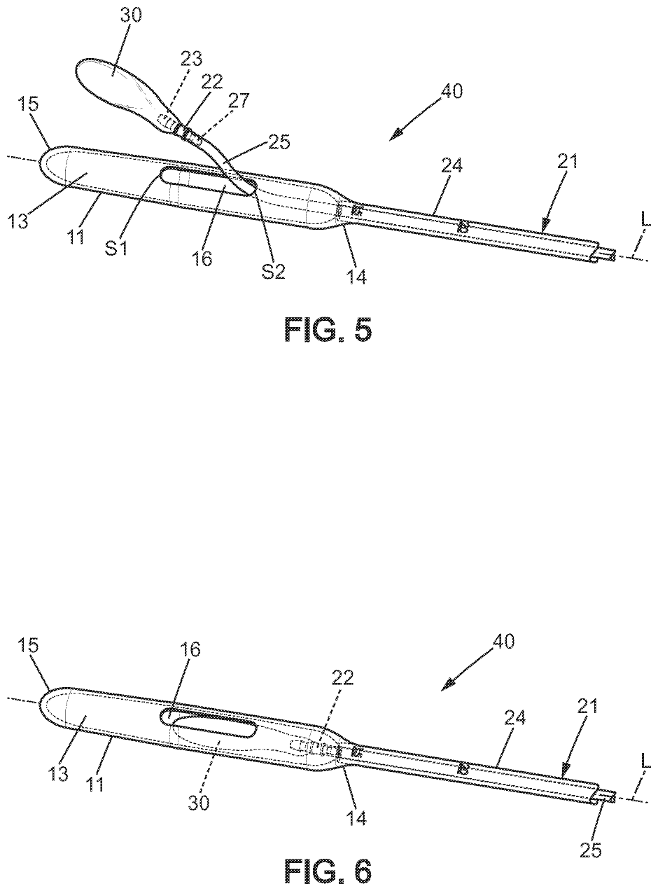

The placement of the gastric balloon 30 on the end piece 22 of the duct 21 is described in connection with FIG. 5. The catheter 25 has been slid in the sheath 24 in such a way that the end piece 22 enters the lateral opening 16 of the casing and exits the cavity 12 of the casing 11. The end piece 22 is thus positioned outside of the cavity 12 of the casing 11.

The gastric balloon 30 is in the deflated state. It is attached to the end piece 22 of the duct 21 via the valve 32.

The catheter 25 can then be slid again in the sheath 24, so that the end piece 22 is positioned in the inflation position, as depicted in FIG. 6. The gastric balloon 30 is housed entirely inside the casing 11.

The placement device 10 is then inserted via the insertion end 15 of the casing 11 into the mouth and the oesophagus of the individual until the casing 11 reaches the stomach of the individual.

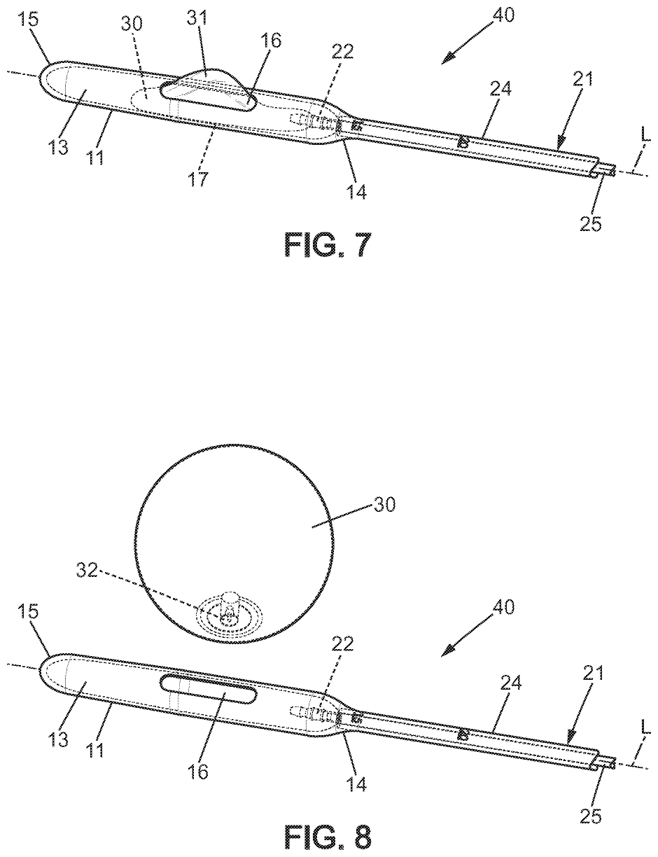

As depicted in FIG. 7, the gastric balloon 30 is then inflated. The duct 21 is supplied at the proximal end 26 of the catheter 25 with a fluid, and the catheter 25 conveys the fluid as far as the gastric balloon 30 via the end piece 22 which collaborates with the valve 32.

The fluid is introduced into the valve 32 in the open state. The fluid in the duct 21 creates a pressure at the end piece 22, which in turn creates a pressure on the valve 32. This opens the valve 32. The gastric balloon 30 can thus receive the fluid and become inflated, to pass from the deflated state to the inflated state.

When the gastric balloon 30 is in the process of being inflated, namely as it is passing from the deflated state to the inflated state, the gastric balloon 30 bears against the bearing surface 17 of the lateral wall 13 of the casing. The bearing surface 17 guides the gastric balloon 30 towards the lateral opening 16. Part 31 of the gastric balloon 30 therefore enters the lateral opening 16 and automatically leaves the casing 11 via the lateral opening 16.

Once the gastric balloon 30 is in the inflated state, it is detached from the end piece 22. The gastric balloon 30 may for example be detached from the end piece 22 as a result of the sliding of the catheter 25 in the sheath 24 allowing the gastric balloon 30 to be brought into contact with the bearing edge formed by the vertex S2 of the lateral opening 16. The gastric balloon 30 is therefore held out of the casing 11 by butting against the bearing edge and the end piece 22 is therefore carried into the sheath 24, which causes the gastric balloon 30 to become detached from the casing 11. The gastric balloon 30 thus finds itself outside of the casing 11 and disconnected from the placement device 10, as has been depicted in FIG. 8. The gastric balloon 30 is thus implanted in the stomach of the individual, in the fundus.

The valve 32 of the gastric balloon 30, in the closed state, prevents the fluid contained in the gastric balloon from leaving the gastric balloon 30, and the balloon remains in the inflated state in the stomach of the individual.

The placement device 10 is then withdrawn from the oesophagus and from the mouth of the individual.

1. Placement device (10) for an inflatable gastric balloon (30) for the placement of the gastric balloon (30) in a stomach, the gastric balloon (30) having a deflated state and an inflated state, the placement device (10) comprising:
an elastically deformable casing (11) delimiting a cavity (12) configured to receive the gastric balloon (30) in the deflated state, the casing (11) extending along a longitudinal axis (L) and comprising a lateral wall (13) extending around the longitudinal axis (L) between a connection end (14) and an insertion end (15),
a duct (21) configured to be supplied with a fluid and to convey the fluid to inflate the gastric balloon (30), the duct (21) being attached to the connection end (14) of the casing (11), and comprising an end piece (22) configured to removably attach the gastric balloon (30),
wherein
the end piece (22) has an inflation position in which said end piece (22) extends into the cavity (12) of the casing (11),
and the lateral wall (13) of the casing (11) has a lateral opening (16) configured to allow the gastric balloon (30) to exit the cavity (12) when said gastric balloon (30) passes from the deflated state to the inflated state.

2. Placement device (10) according to claim 1, wherein the duct (21) comprises a sheath (24) attached to the connection end (14) of the casing (11) and a catheter (25) extending inside the sheath (24), the catheter (25) being mounted with the ability to slide with respect to the sheath (24) and comprising a proximal end (26) configured to be supplied with fluid and a distal end (27), the distal end (27) comprising the end piece (22) and projecting from the sheath (24) in the inflation position.

3. Placement device (10) according to claim 1, wherein the lateral wall (13) of the casing (11) has a bearing surface (17), opposite the lateral opening (16) and configured so that the gastric balloon (30) bears against said bearing surface (17) when said gastric balloon (30) passes from the deflated state to the inflated state.

4. Placement device (10) according to claim 1, wherein the lateral opening (16) of the casing (11) has a bearing edge configured so that the gastric balloon (30) bears against said bearing edge for detaching the gastric balloon (30) from the end piece (22).

5. Placement device (10) according to claim 1, wherein, in the inflation position, the end piece (22) extends along the longitudinal axis (L) and is positioned between the connection end (14) of the casing (11) and the lateral opening (16) of the casing (11) along the longitudinal axis L.

6. Placement device (10) according to claim 1, specially designed for a gastric balloon (30) comprising a valve (32) configured to collaborate with the end piece (22) of the duct (21) and having an open state allowing fluid to pass and a closed state blocking the fluid, and wherein the end piece (22) is configured to be push-fitted removably into the valve (32) of the gastric balloon (30).

7. Placement device (10) according to claim 6, wherein the end piece (22) has an end piece axis and comprises at least one shoulder (23) extending transversely with respect to the end piece axis.

8. Placement device (10) according to claim 1, wherein the lateral opening (16) has a shape that is oblong along the longitudinal axis (L).

9. Placement device (10) according to claim 1, specially designed for a spherical gastric balloon (30) having a diameter D in the inflated state, and wherein the cavity (12) of the casing (11) and the lateral opening (16) each have a length (L12, L16) considered along the longitudinal axis (L) and a width (I12, I16) considered perpendicular to the longitudinal axis (L) such that:
a ratio of the diameter D of the gastric balloon (30) to the length (L12) of the cavity (12) of the casing (11) is comprised between 0.44 and 0.83,
a ratio of the diameter D of the gastric balloon (30) to the width (I12) of the cavity (12) of the casing (11) is comprised between 3.2 and 6.7,
a ratio of the length (L16) of the lateral opening (16) of the casing (11) to the length (L12) of the cavity (12) of the casing (11) is comprised between 0.17 and 0.67,
a ratio of the width of the lateral opening of the casing to the width of the cavity of the casing is comprised between 0.24 and 0.67.

10. Placement device (10) according to claim 9, specially designed for a spherical gastric balloon (30) having a diameter D comprised between 8 cm and 10 cm in the inflated state, wherein:
the length (L12) of the cavity (12) of the casing (11) is comprised between 12 cm and 18 cm, and the width (I12) of the cavity (12) of the casing (11) is comprised between 1.5 cm and 2.5 cm,
the length (L16) of the lateral opening (16) of the casing (11) is comprised between 3 cm and 8 cm and the width (I16) of the lateral opening (16) of the casing (11) is comprised between 0.6 cm and 1 cm.

11. Placement device (10) according to claim 1, wherein the casing (11) is a cylinder along the longitudinal axis (L), of circular cross section.

12. Placement device (10) according to claim 1, wherein the insertion end (15) of the casing (11) is convex towards the outside of the cavity (12), so that the placement device (10) can be introduced into a natural tract of the human body.

13. System (40) for treating obesity in an individual, comprising:
a placement device (10) according to claim 1, and
an inflatable gastric balloon (30) having an inflated state and a deflated state.

14. System (40) for treating obesity in an individual according to claim 15, wherein the gastric balloon (30) comprises a valve (32) configured to collaborate with the end piece (22) of the duct (21) and having an open state allowing fluid to pass and a closed state blocking the fluid.

15. Placement device (10) according to claim 12, wherein the insertion end (15) of the casing (11) is hemispherical.
